(19) United States
(12) Patent Application Publication
(10) Pub. No.: US 2026/0140115 A1
(43) Pub. Date: May 21, 2026

(54) COMPLEMENT PROTEIN C8 GAMMA BIOMARKERS FOR DETECTING AND MONITORING CANCERS

(71) Applicant: Sun Jet Biotechnology Inc., Taipei City (TW)

(72) Inventor: Yeou-Guang Tsay, Taipei (TW)

(73) Assignee: Sun Jet Biotechnology Inc., Taipei City (TW)

(21) Appl. No.: 19/348,016

(22) Filed: Oct. 2, 2025

Related U.S. Application Data
(60) Provisional application No. 63/721,650, filed on Nov. 18, 2024.

(51) Int. Cl. G01N 33/574

(52) U.S. Cl. CPC G01N 33/5752; G01N 2333/4716

(57) ABSTRACT

Disclosed is a method for diagnosing a cancer health state, or a change in cancer health state in a patient, or for diagnosing a risk of the change or presence of a cancer in a patient, comprising determining, in a plasma sample from said patient, one or more biomarker values that correspond to complement protein C8 gamma-containing complex structures, and assigning the patient as having or not having cancer, or having or not having a change in cancer health state, or having or not having a risk of cancer based on said biomarker values, wherein said cancer is preferably lung cancer (LC).

### FIELD OF THE INVENTION

The present invention pertains to a biomarker and a use of biomarker based on complement protein C8 gamma-containing complex structures for detecting and monitoring cancers.

### BACKGROUND OF THE INVENTION

Lung cancer is a type of malignant lung tumor that occurs when cell growth in the lung is uncontrolled. Carcinomas, which account for over 98% of lung cancers, are generally classified into two types based on the type of cell that initiates the tumor: small-cell lung carcinoma (SCLC) and non-small-cell lung carcinoma (NSCLC). Smoking is responsible for 85% of lung cancer cases, while other risk factors include genetic predisposition, exposure to radon gas, asbestos, or polluted air. SCLC is the more aggressive type of lung cancer, characterized by rapid spread, and the main risk factor is tobacco smoking. In contrast, NSCLC has been on the rise in recent years, particularly among non-smokers. The most common types of NSCLC are adenocarcinoma, squamous cell carcinoma, and large cell carcinoma. The former two originate from mucus-producing cells lining the smallest and larger airways, respectively, while the latter originates from neuroendocrine cells in the lung.

Over 80% of lung cancer (LC) patients who survive more than five years are those with early-stage disease, while high mortality is seen in patients with advanced disease. Although early-stage LC markers should improve prognosis, current clinical biomarkers are neither sensitive nor specific enough. Thus far, there are no FDA-approved plasma biomarkers for early LC detection. CEA (carcinoembryonic antigen) and CYFRA21-1 (cytokeratin-19 fragment) are biomarkers for lung cancer diagnosis and monitoring used by some hospitals. CEA is a glycoprotein that is normally produced during fetal development but is also expressed in the plasma in other types of cancers. High levels of CEA are most commonly found in cases of colorectal adenocarcinoma, but it is now considered an auxiliary biomarker for the monitoring or diagnosis of cancer due to its wide expression in multiple diseases. CYFRA21-1, a fragment of cytokeratin-19, is commonly found in epithelial cells, including those in the lung, and is more specific for squamous cell carcinoma than for other types of lung cancer. Nevertheless, various cancers or benign lung diseases may also cause an increase in CYFRA21-1. Therefore, these biomarkers are typically combined with other imaging methods.

Overall, there remains an urgent need for biomarkers that demonstrate superior all-around performance in lung cancer screening. Protein complexes are actual structures that carry biological functions. However, accurately quantifying these protein complexes in conventional western blotting is challenging due to issues with transfer efficiency and operational inconsistencies.

### SUMMARY OF THE INVENTION

The inventors surprisingly found that different species of complement protein C8 gamma complex structures (or multimeric structures) can be detected and quantified from human plasma sample, and that their levels and ratios are indicative of the risk of cancers including lung cancer (LC).

In one aspect, the present invention provides a method for diagnosing a cancer health state in a patient, comprising: determining, in a plasma sample from said patient, one or more biomarker values that correspond to complement protein C8 gamma-containing complex structures; and assigning the patient as having or not having cancer, or having or not having a change in cancer health state, or having or not having a risk of cancer based on said biomarker values, wherein said cancer is preferably lung cancer (LC).

In one further aspect, the present invention provides a method for diagnosing a change in cancer health state in a patient, comprising: determining, in a plasma sample from said patient, one or more biomarker values that correspond to complement protein C8 gamma-containing complex structures; and assigning the patient as having or not having cancer, or having or not having a change in cancer health state, or having or not having a risk of cancer based on said biomarker values, wherein said cancer is preferably lung cancer (LC).

In one yet aspect, the present invention provides a method for diagnosing a risk of the change or presence of a cancer in a patient, comprising: detecting, in a plasma sample from said patient, one or more biomarker values that correspond to complement protein C8 gamma-containing complex structures; and assigning the patient as having or not having cancer, or having or not having a change in cancer health state, or having or not having a risk of cancer based on said biomarker values, wherein said cancer is preferably lung cancer (LC).

In one further aspect, the present invention provides use of a complement protein C8 gamma-containing complex structure as a biomarker for cancer, including lung cancer (LC).

In one yet aspect, the present invention provides a capture reagent for complement protein C8 gamma or a complement protein C8 gamma-containing complex structure, for use in diagnosing (in vitro) a cancer health state in a patient, wherein said cancer is preferably lung cancer (LC).

Specifically, the use comprises determining one or more biomarker values that correspond to complement protein C8 gamma-containing complex structures using the capture reagent for complement protein C8 gamma or a complement protein C8 gamma-containing complex structure.

Also provided is a kit for performing the method as described herein, comprising a capture reagent for complement protein C8 gamma or a complement protein C8 gamma-containing complex structure, and instructions for performing the method.

In some embodiments, the one or more biomarker values are determined by performing an in vitro assay. The in vitro assay may be an immunoassay, including but not limited to a western blotting and a capillary electrophoresis.

In some embodiments, determining the biomarker values comprises performing an in vitro assay, wherein said in vitro assay comprises a capture reagent for complement protein C8 gamma or a complement protein C8 gamma-containing complex structure.

In some embodiments, the capture reagent is an antibody.

In some embodiments, the one or more biomarker values are determined by performing a capillary electrophoresis under non-reducing conditions. According to certain preferred embodiments, the one or more biomarker values include IP₈₀, IP₁₂₅, or a combination thereof. According to certain preferred embodiments, the assigning is based on

$$\frac{{IP}_{125}}{{IP}_{80}} .$$

It is to be understood that both the foregoing general description and the following detailed description are exemplary and explanatory only and are not restrictive of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1 shows the results of western blotting for analyzing plasma complement protein C8 gamma (C8G) species from five healthy subjects (HS) and five lung cancer (LC) patients under non-reducing conditions. The numbers to the left indicate the sizes of molecular mass markers (kDa) at the marked positions.

FIG. 2 shows the migration profiles of plasma C8G species in capillary western analyses for 32 healthy subjects and 86 lung cancer patients. All of the patients have disease severity no higher than stage 2. The left numbers indicate signal percentages relative to the ˜40-kDa peak, while the bottom numbers denote the migration positions of proteins corresponding to their molecular masses. The pair of numbers at the top of the shaded box for P₄₀, P₈₀, or P₁₂₅ indicates the mass range boundaries used for summing signals.

FIG. 3 shows the box plots illustrating the statistical analysis of the C8G indices in patients with lung cancer. The statistical analyses of two C8G multimeric indices were performed. Each dot in the plot represents the index value for an individual subject, with the mean indicated for each group.

FIG. 4 shows the receiver operating characteristic (ROC) curves of the C8G indices distinguishing between healthy individuals and LC patients. Sensitivity is represented on the vertical axis, and (1−specificity) is represented on the horizontal axis for each C8G index cutoff value. Each dot in the plot corresponds to the point with the maximum (sensitivity+specificity). The specific cutoff values are marked by dots, with corresponding performance indicated using dotted lines in parallel with the axes.

FIG. 5 shows a summary of the results of two C8G indices for all healthy subjects (HS) and LC patients in this study. Dark grey shading indicates values that are two times higher than the cutoff, whereas light grey shading represents values falling between one and two times the cutoff.

### DETAILED DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereto known to those skilled in the art.

The term “biomarker” used herein refers to a measurable characteristic, either within or external to an organism, that indicates a specific physiological state or the presence of a disease. Biomarkers can be used as indicators for assessing physiological processes, disease progression, drug response, or treatment effectiveness. They may include molecules, cells, tissues, physiological indicators, or imaging features, with their changes often closely associated with disease occurrence, progression, treatment response, etc. Biomarkers have significant applications in clinical diagnosis, prediction, monitoring, and treatment, aiding in improving the accuracy of early disease detection, diagnosis, prognosis assessment, as well as evaluating the effectiveness and safety of treatment regimens.

The term “cancer” used herein refers to a group of diseases characterized by the uncontrolled growth and spread of abnormal cells. These cells can invade and destroy surrounding healthy tissues and can also metastasize to distant parts of the body. Cancer can arise from almost any type of cell in the body and may develop in various organs and tissues. It is typically caused by genetic mutations or other factors that disrupt the normal regulation of cell growth and division.

A biomarker value for the biomarkers described herein can be determined using any of a variety of known analytical methods. In some embodiments, the biomarker value can be determined through performing an in vitro assay, for example, an immunoassay. In one embodiment, the determination of a biomarker value involves the use of a capture reagent. A biomarker value may also refer to a ratio calculated based on two or more biomarker values, e.g.,

$$\frac{{IP}_{125}}{{IP}_{80}} .$$

As used herein, a “capture agent” or “capture reagent” refers to a molecule that is capable of binding specifically to a biomarker. Capture reagents include but are not limited to aptamers, antibodies, antigens, adnectins, ankyrins, other antibody mimetics and other protein scaffolds, autoantibodies, chimeras, small molecules, an F(ab′)₂ fragment, a single chain antibody fragment, an Fv fragment, a single chain Fv fragment, a nucleic acid, a lectin, a ligand-binding receptor, affibodies, nanobodies, imprinted polymers, avimers, peptidomimetics, a hormone receptor, a cytokine receptor, and synthetic receptors, and modifications and fragments of these.

Complement protein C8 gamma, encoded by the C8G gene, is a member of the lipocalin family and one of the subunits of the membrane attack complex in the complement system. C8 binds to the C5B-7 complex, forming the C5B-8 complex, which catalyzes the polymerization of C9. C8 is composed of three subunits: alpha, beta, and gamma. The gamma subunit is linked to the alpha subunit by a disulfide bond. The C8 gamma-alpha heterodimer is non-covalently associated with the beta subunit (Schreck S F, Parker C, Plumb M E, Sodetz J M., Biochim Biophys Acta. 1482(1-2):199-208 (2000)).

The terms “complement protein C8 gamma multimer,” “complement protein C8 gamma-containing complex structure,” and “complement protein C8 gamma complex structure” are used interchangeably herein and refer to a protein complex comprising at least one complement protein C8 gamma subunit, wherein the at least one complement protein C8 gamma subunit may be linked to one or more partners (proteins or polypeptides other than complement protein C8 gamma).

In one aspect, the present invention provides a method for diagnosing a cancer health state, or a change in cancer health state in a patient, or for diagnosing a risk of the change or presence of a cancer in a patient, comprising determining, in a plasma sample from said patient, one or more biomarker values that correspond to complement protein C8 gamma-containing complex structures, and assigning the patient as having or not having cancer, or having or not having a change in cancer health state, or having or not having a risk of cancer based on said biomarker values, wherein said cancer is preferably lung cancer (LC).

In another aspect, the present invention provides use of a complement protein C8 gamma-containing complex structure as a biomarker for cancers, including LC.

The present invention also provides a capture reagent for complement protein C8 gamma or a complement protein C8 gamma-containing complex structure, for use in diagnosing (in vitro) a cancer health state in a patient. Said use may comprise determining one or more biomarker values that correspond to complement protein C8 gamma-containing complex structures using the capture reagent for complement protein C8 gamma or a complement protein C8 gamma-containing complex structure.

In one further aspect, the present invention provides a kit for performing the method as described herein, comprising a capture reagent for complement protein C8 gamma or a complement protein C8 gamma-containing complex structure, and instructions for performing the method.

In one further aspect, the present invention provides use of a capture reagent for complement protein C8 gamma or a complement protein C8 gamma-containing complex structure in the preparation of a kit for performing the method as described herein.

According to the present invention, a patient may be assigned as having or not having a cancer, or having or not having a change in a cancer health state, or having or not having a risk of a cancer, based on a higher biomarker value that corresponds to a complement protein C8 gamma-containing complex structure, or a lower biomarker value that corresponds to a complement protein C8 gamma-containing complex structure.

As used herein, a higher (biomarker) value or lower (biomarker) value can refer to a value that is higher or lower compared with a reference level. For example, a lower value can be lower than a reference level by at least 30%, 40%, 50%, 60%, 70%, 80%, 90% or 100%; and higher value can be higher than a reference level by at least 30%, 40%, 50%, 60%, 70%, 80%, 90% or 100%. In some embodiments, a reference level can be a standard (or a threshold) value in a normal individual or a control group. For example, a standard or threshold value can be set based on an average or median level obtained from a cohort of normal subjects. In some embodiments, the cohort of subjects can be a population of normal human subjects (without cancer, or without a cancer). In addition, a threshold value can be set further based on a desired sensitivity and/or specificity for detecting or diagnosing a cancer.

According to certain embodiments of the present invention, three species or groups of human plasma complement protein C8 gamma-containing proteins can be resolved using conventional SDS-PAGE and western blotting under non-reducing conditions, including: (i) 71-, 72.5-, 74- and 75.1-kDa species, sizes of which are consistent with that for C8 alpha-C8 gamma heterodimer, (ii) a high-MW species with molecular mass of 143 kDa, and (iii) 44 and 48 kDa species.

According to the present invention, human plasma complement protein C8 gamma complex structures can also be resolved by a capillary gel electrophoresis into three major peaks, including P₈₀, P₁₂₅, and P₄₀ and correspond to the 71-, 72.5-, 74- and 75.1-kDa species, high-MW species, and 44 and 48 kDa species, respectively.

A biomarker value is indicative of a concentration of a biomarker, or a ratio of concentrations of the biomarkers in a sample. A biomarker value of the present invention may be a signal intensity or normalized signal intensity of any of the peaks P₈₀ and P₁₂₅, denoted as IP₈₀ and IP₁₂₅, respectively, or a ratio of the signal intensities. The signal intensity can be measured as the area under the peak. In some embodiments, the signal intensity is measured as the area under the peak in an immunodetection.

In some embodiments, the one or more biomarker values are determined through performing a capillary electrophoresis under non-reducing conditions. Specifically, biomarker signals are detected by performing the capillary electrophoresis and immunodetection, and then the one or more biomarker values are determined based on the detected biomarker signals. According to certain preferred embodiments, the one or more biomarker values include IP₈₀, IP₁₂₅, or a combination thereof. According to certain preferred embodiments, based on

$$\frac{{IP}_{125}}{{IP}_{80}} ,$$

the patient is assigned as having or not having a cancer, or having or not having a change in a cancer health state, or having or not having a risk of a cancer.

The present invention is further illustrated by the following examples, which are provided for the purpose of demonstration rather than limitation.

### Examples

### 1. Materials and Methods

### 1.1 Reagents

Immobilon Western Chemiluminescent HRP Substrate (Millipore; Cat. No. WBKLS0500) was for signal development in western blotting.

### 1.2 Clinical Samples

Plasma samples from lung cancer (LC) patients (IRB: EC1120809-F-E) were obtained from the National Health Research Institutes Biobank. The distribution of pathologies among LC patients was established to reflect clinical incidence rates: 60% adenocarcinoma, 20% squamous cell carcinoma, 15% small-cell carcinoma, and 5% combined for males; and 85% adenocarcinoma, 5% squamous cell carcinoma, 5% small-cell carcinoma, and 5% combined for females. Blood samples were processed into plasma following the guidelines of the National Health Research Institutes Biobank. The supernatant, representing the plasma fraction, was stored at −80° C. until needed for further analysis.

### 1.3 Western Blot Analysis

For each well, 0.3 μL of plasma sample was combined with SDS-PAGE sample dye, consisting of 0.04 M Tris-HCl (pH 6.8), 1 M glycerol, 0.05 M SDS, and bromophenol blue. The mixture was with or without addition of 0.3 μL of β-mercaptoethanol for reducing and non-reducing analyses, respectively. After incubating at 95° C. for 5 minutes, the sample mixture was loaded into wells of a 4% stacking and 12% separating Tris-based polyacrylamide gel. Following SDS-PAGE, the proteins were transferred to a nitrocellulose membrane using CAPS buffer (0.02 M 3-(cyclohexylamino)-1-propanesulfonic acid in 10% methanol, pH 11). The membrane was blocked with 1% BSA in TBST buffer (0.02 M Tris, 0.14 M NaCl, 0.1% Tween 20, pH 7.6) and then incubated overnight at 4° C. with anti-complement protein C8 gamma (Abcam, ab181182, rabbit-derived, diluted 1:1,000). Afterward, it was treated with an anti-rabbit HRP conjugate (Jackson, donkey-derived, 711-035-152, diluted 1:10,000) for 1 hour at room temperature, followed by a wash with TBST. Chemiluminescent signals were generated using horseradish peroxidase-conjugated secondary antibodies and detected with an LAS-4000 imaging system (Fujifilm, Japan).

### 1.4 Automated Capillary Electrophoresis-Immunodetection

Reagents were obtained from BioTechne, USA, unless otherwise specified. Plasma samples were diluted 1:200, and 5× Fluorescent Master Mix along with 0.76 ng of recombinant complement protein C8 gamma (Cusabio, CSB-EP004196HU) were added to each sample. The samples were incubated at 37° C. for 30 minutes in a water bath. Six microliters of each sample were then loaded into the top-row wells of plates preloaded with proprietary electrophoresis buffers designed for protein separation in the range of 12 to 230 kDa. The remaining rows of the plate were filled with 1% bovine serum albumin (Bionovas, AA0530-0250) to serve as an antibody diluent.

The primary and secondary antibody solutions, chemiluminescence reagents, and wash buffer were used according to the manufacturer's instructions. For the biotinylated SimpleWestern size standard, these rows were filled with antibody diluent and streptavidin-HRP (Genetex, GTX27403) instead of the primary and secondary antibody solutions. The primary antibody used was anti-complement protein C8 gamma (Abcam, ab181182, rabbit-derived, diluted 1:1,000). The secondary antibody, anti-rabbit HRP conjugate (Jackson, donkey-derived, 711-035-152, diluted 1:2,000), was also prepared in the antibody diluent.

The plates were centrifuged for 5 minutes at 1,000 g at room temperature. Subsequently, the plates and capillaries were loaded into a SimpleWestern™ system (BioTechne USA), and assays were performed using the standard protocol for protein separation in the 12- to 230-kDa range, as specified in version 6.1.0 of the accompanying Compass software. The separation time was set to the default of 25 minutes, and the separation voltage was set to 375 V. Data were reported by the Compass software as chemiluminescence signals versus apparent molecular weight (MW). Apparent MWs were determined by aligning size marker peaks with capillary positions and utilizing signals from fluorescently labeled protein standards within the 5× Master Mix to account for variations in migration among capillaries.

### 1.5 Migration Profile Analysis

Data analysis collected through capillary electrophoresis utilized Microsoft Excel 2021 Visual Basic for Applications (VBA). Raw data obtained from the Compass for SW software were converted to text format and transformed into graphs, with the x-axis representing log-scaled molecular weight and the y-axis depicting the intensity of the immunodetection signals. To identify specific mass ranges, we developed an in-house peak detection and integration program, which was subsequently validated through manual confirmation. The identified ranges were then used to calculate the total areas of the specified peaks. Sensitivities and specificities for different cutoff values were characterized using receiver operating characteristic (ROC) curve analyses.

### 2. Results

### 2.1 in Lung Cancer Patients, Circulating Complement Protein C8 Gamma-Containing ˜143-kDa Multimeric Species Showed Significant Increases Compared to the 71-74 kDa Species

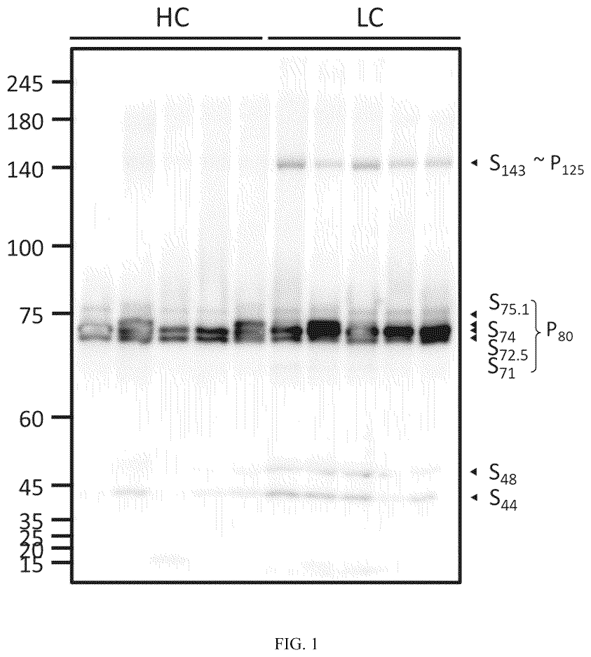

To investigate how significant complement protein C8 gamma multimeric structures changes in the lung patient group, we conducted western blot analyses following conventional SDS-PAGE under non-reducing conditions (FIG. 1). For healthy controls, there were a number of C8 gamma-containing species that have small differences in their sizes, including 71-, 72.5-, 74- and 75.1-kDa species, and the smaller three ones are predominant. Intriguingly, some have all three major isoforms, and the other have only the smaller 71- and 72.5-kDa ones (FIG. 1). The sizes of these species are consistent with that for C8 alpha-C8 gamma heterodimer with interchain disulfide between Cys-194 in C8 alpha and Cys-60 in C8 gamma.

The same SDS-PAGE analyses of samples from LC patients showed that, while these C8 gamma species were preserved, there were two groups of protein bands detected in most patients. The first is a high-MW species with the molecular mass of 143 kDa. The second had two isoforms, with molecular masses of 44 and 48 kDa. The prevalence of these isoforms in lung cancer patients appears to be quite high, as five out of five tested samples contained these species. The C8 gamma-containing structures with the indicated sizes are not documented in the literature reviewed. On the other hand, there is currently no direct evidence or reports linking C8 gamma to malignant diseases, such as lung cancer. Thus, our findings offer the valuable evidence regarding the presence of the aforementioned C8 gamma variants in lung cancer patients.

2.2 for Lung Cancer Patients, Peak P₁₂₅ Resolved Using Capillary Electrophoresis had a Significant Increase Relative to the P₈₀ Species

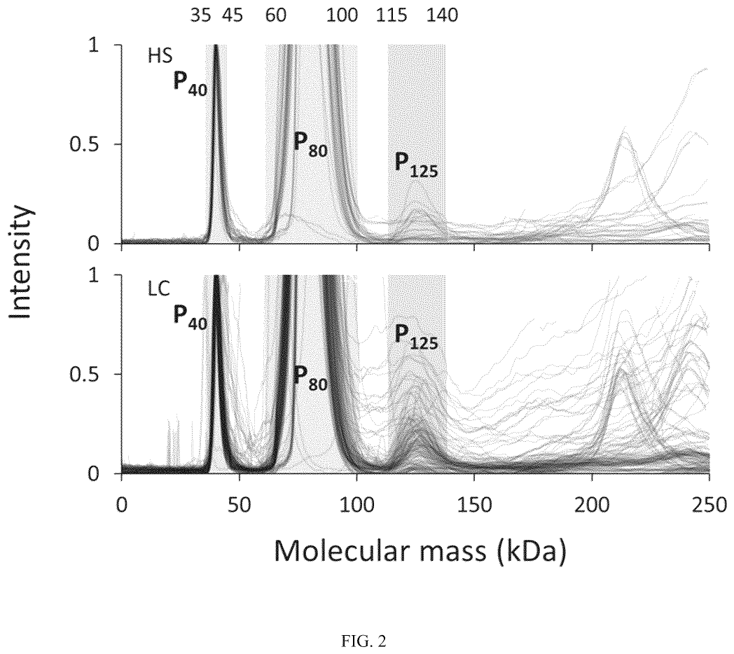

To better quantitatively determine how these C8 gamma multimeric structures changed in cancer patients, we took advantage of an automated western blotting system to analyze plasma samples under non-reducing conditions. For healthy subjects, this system resolved the putative C8 alpha-C8 gamma heterodimer as the peak at 80 kDa (i.e. Pa). Due to the limited resolution of capillary electrophoresis, the four structures seen in conventional electrophoresis could become this peak alone (FIGS. 1 and 2).

Since the staining signals for LC patients appeared to be a little higher in regular western blotting, we decided to introduce a recombinant C8 gamma protein as the internal control to quantitatively analyze peak P₈₀. This amount standard migrated at the position of ˜40 kDa, consistent with its expected size due to the in-frame fusion to the His-SUMO tag at its N-terminus. In short, this standard protein gives the peak P₄₀. Later analyses, however, showed that there was little difference for P₈₀ intensities (IP₈₀) between controls and LC patients (data not shown).

For LC patients, almost all of them had one additional peak detected at the position of 125 kDa, i.e. peak P₁₂₅, which, similar to 143-kDa isoform in SDS-PAGE, was barely seen in healthy controls (FIG. 2). We next determined the mass ranges to sum the signals to acquire IP₄₀, IP₈₀ and IP₁₂₅ values. As mentioned above, IP₄₀ comes from added amount marker, which has used a reference, like IP₈₀, to gauge the IP₁₂₅ increase. Thus, two indices

$$\frac{{IP}_{125}}{{IP}_{80}} and \frac{{IP}_{125}}{{IP}_{40}}$$

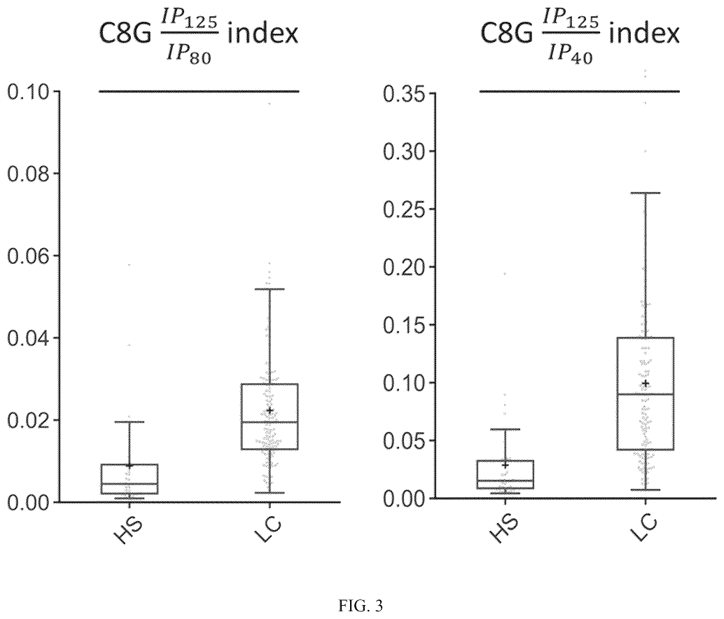

have been implemented, and the values for 86 stage I and II LC patients and 32 healthy controls have been determined. For healthy subjects, the values averaged around 0.01 and 0.03, respectively, while the means of these two for LC patients were 0.02 and 0.10 (FIG. 3). The box plots also showed that it is possible to draw a cutoff to distinguish LC patients from healthy subjects (FIG. 3).
2.3 C8 Gamma Multimeric Indices Based on IP₁₂₅ Performed Well in Distinguishing LC Patients from Healthy Controls

As the quartile analyses revealed a good distinction between healthy controls and cancer patients, we used receiver operating characteristic (ROC) curves to investigate the best cutoff values for the two indices to distinguish patients from healthy controls. Overall, both indices

$$\frac{{IP}_{125}}{{IP}_{80}} and \frac{{IP}_{125}}{{IP}_{40}}$$

had curves that were closer to the perfect classifier points than to the random classifier lines. The indices

$$\frac{{IP}_{125}}{{IP}_{80}} and \frac{{IP}_{125}}{{IP}_{40}}$$

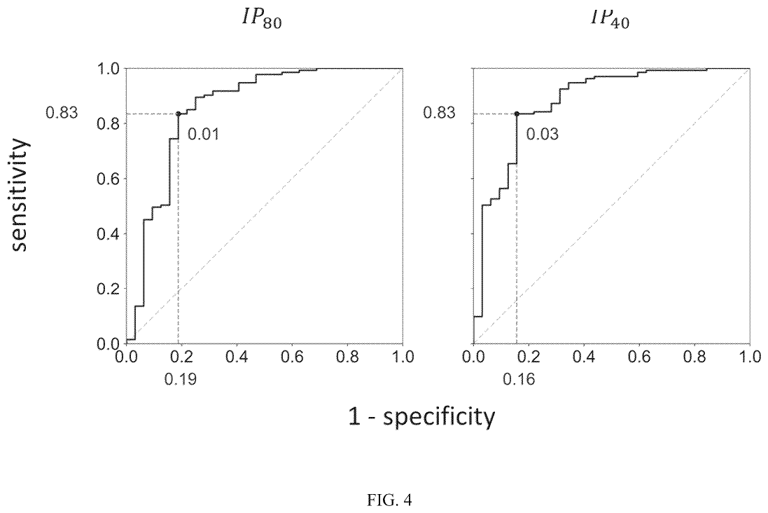

demonstrated remarkable performance in identifying LC patients with a sensitivity of 87% and 79% and a specificity of 75% and 84%, respectively, with the cutoff values of 0.01 and 0.03 (FIG. 4). These results also suggest that the inclusion of the amount marker does not have an impact on the performance of these multimeric indices.

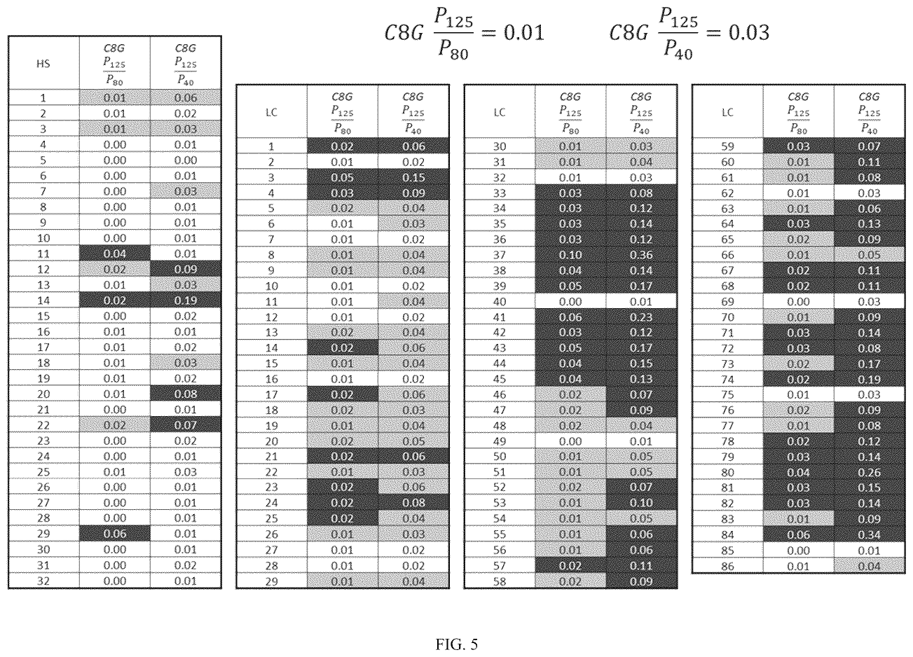

With the indicated cutoffs, all index values were listed for all tested subjects. The performances of these two indices are quite similar, as we can identify ˜84% of LC patients at stage I to II (FIG. 5). Our results showed that higher index values are associated with LC progression, although there is an apparent drop for stage IV diseases (data not shown). Overall, these findings highlight the efficacy of these biomarker indices in high-sensitivity screening of cancer patients.

While this specification contains many specifics, these should not be construed as limitations on the scope of the invention or of what may be claimed, but rather as descriptions of features specific to particular embodiments or examples of the invention. Certain features that are described in this specification in the context of separate embodiments or examples can also be implemented in combination in a single embodiment.

1. A method for diagnosing a cancer health state in a patient, comprising:
determining, in a plasma sample from said patient, one or more biomarker values that correspond to complement protein C8 gamma-containing complex structures; and
assigning the patient as having or not having cancer, or having or not having a change in cancer health state, or having or not having a risk of cancer based on said biomarker values,
wherein said cancer is preferably lung cancer (LC).

2. The method of claim 1, wherein determining the biomarker values comprises performing an in vitro assay, wherein said in vitro assay comprises a capture reagent for complement protein C8 gamma or a complement protein C8 gamma-containing complex structure.

3. The method of claim 2, wherein the capture reagent is an antibody.

4. The method of claim 2, wherein the in vitro assay is a capillary electrophoresis under non-reducing conditions.

5. The method of claim 1, wherein the one or more biomarker values include IP₈₀, IP₁₂₅, or a combination thereof.

6. The method of claim 1, wherein the assigning is based on
$$\frac{{IP}_{125}}{{IP}_{80}} .$$

7. A method for diagnosing a risk of the change or presence of a cancer in a patient, comprising:
determining, in a plasma sample from said patient, one or more biomarker values that correspond to complement protein C8 gamma-containing complex structures; and
assigning the patient as having or not having cancer, or having or not having a change in cancer health state, or having or not having a risk of cancer based on said biomarker values,
wherein said cancer is preferably lung cancer (LC).

8. The method of claim 7, wherein determining the biomarker values comprises performing an in vitro assay, wherein said in vitro assay comprises a capture reagent for complement protein C8 gamma or a complement protein C8 gamma-containing complex structure.

9. The method of claim 8, wherein the capture reagent is an antibody.

10. The method of claim 8, wherein the in vitro assay is a capillary electrophoresis under non-reducing conditions.

11. The method of claim 7, wherein the one or more biomarker values based on IP₈₀, IP₁₂₅, or a combination thereof.

12. The method of claim 7, wherein the assigning is based on
$$\frac{{IP}_{125}}{{IP}_{80}} .$$

13. A kit for performing a method for diagnosing a cancer health state in a patient, comprising a capture reagent for complement protein C8 gamma or a complement protein C8 gamma-containing complex structure, and instructions for performing the method, wherein said cancer is preferably lung cancer (LC).

14. The kit of claim 13 wherein the method comprising determining one or more biomarker values that correspond to complement protein C8 gamma-containing complex structures using the capture reagent, and assigning the patient as having or not having cancer, or having or not having a change in cancer health state, or having or not having a risk of cancer based on said biomarker values.

15. The kit of claim 13, wherein the capture reagent is an antibody.

16. The kit of claim 14, wherein determining the biomarker values comprises performing an in vitro assay using the capture reagent.

17. The kit of claim 16, wherein the in vitro assay is a capillary electrophoresis under non-reducing conditions.

18. The kit of claim 13, wherein the one or more biomarker values include IP₈₀, IP₁₂₅, or a combination thereof.

19. The kit of claim 13, wherein the assigning is based on
$$\frac{{IP}_{125}}{{IP}_{80}} .$$
